(19) United States
(12) Patent Application Publication
(10) Pub. No.: US 2008/0255184 A1
(43) Pub. Date: Oct. 16, 2008

(54) Thienopyridine B-Raf Kinase Inhibitors

(75) Inventor: Jun Tang, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SMITHKLINE BEECHAM CORPORATION

(21) Appl. No.: 12/090,575

(22) PCT Filed: Oct. 23, 2006
(86) PCT No.: PCT/US06/60145

§ 371 Date: Apr. 17, 2008

Related U.S. Application Data
(60) Provisional application No. 60/733,566, filed on Nov. 4, 2005.

(51) Int. Cl. A61K 31/444; C07D 495/04; A61P 35/00

(52) U.S. Cl. 514/301; 546/114

(57) ABSTRACT

The present invention provides thienopyridine compounds, compositions containing the same, as well as processes for the preparation and their use as pharmaceutical agents.

### FIELD OF THE INVENTION

The present invention relates to thienopyridine derivatives, compositions and medicaments containing the same, as well as processes for the preparation and use of such compounds, compositions and medicaments. Such pyridine derivatives are useful in the treatment of diseases associated with inappropriate B-Raf kinase activity.

### BACKGROUND OF THE INVENTION

Raf protein kinases are key components of signal transduction pathways by which specific extracellular stimuli elicit precise cellular responses in mammalian cells. Activated cell surface receptors activate ras/rap proteins at the inner aspect of the plasmamembrane which in turn recruit and activate Raf proteins. Activated Raf proteins phosphorylate and activate the intracellular protein kinases MEK1 and MEK2. In turn, activated MEKs catalyse phosphorylation and activation of p42/p44 mitogen-activated protein kinase (MAPK). A variety of cytoplasmic and nuclear substrates of activated MAPK are known which directly or indirectly contribute to the cellular response to environmental change. Three distinct genes have been identified in mammals that encode Raf proteins; A-Raf, B-Raf and C-Raf (also known as Raf-1) and isoformic variants that result from differential splicing of mRNA are known.

Inhibitors of Raf kinases have been suggested for use in disruption of tumor cell growth and hence in the treatment of cancers, e.g. histiocytic lymphoma, lung adenocarcinoma, small cell lung cancer and pancreatic and breast carcinoma; and also in the treatment and/or prophylaxis of disorders associated with neuronal degeneration resulting from ischemic events, including cerebral ischemia after cardiac arrest, stroke and multi-infarct dementia and also after cerebral ischemic events such as those resulting from head injury, surgery and/or during childbirth.

The present inventors have discovered novel thienopyridine compounds, which are inhibitors of B-Raf kinase. Such derivatives are useful in the treatment of disorders associated with inappropriate B-Raf kinase activity.

### SUMMARY OF THE INVENTION

In a first aspect of the present invention, there is provided a compound of formula (I):

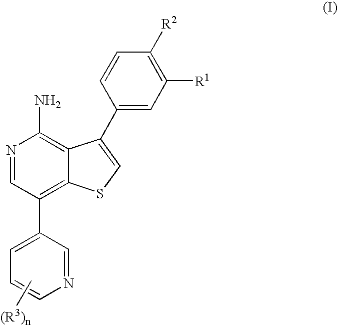

wherein:
R¹ is —N(H)—C(O)R⁴ or —N(H)—C(O)—N(H)R⁴ and R² is H; or
R¹ and R² together with the phenyl ring to which they are bonded form a moiety (i);

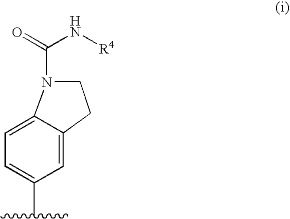

each R³ is independently selected from the group consisting of halo, alkyl, and haloalkyl;
R⁴ is selected from the group consisting of cyclohexyl; benzyl; unsubstituted, monosubstituted, or disubstituted phenyl wherein the substituent(s) is independently selected from the group consisting of halo, alkyl, haloalkyl, alkoxy, haloalkoxy and benzyloxy; methylene-thienyl; and dimethyl-4-isoxazolyl; and
n is 0, 1, or 2;
   or a pharmaceutically acceptable salt or solvate thereof.

In a second aspect of the present invention, there is provided a pharmaceutical composition comprising a compound of formula (I) or a pharmaceutically acceptable salt or solvate thereof. In one embodiment, the pharmaceutical composition further comprises one or more of pharmaceutically acceptable carriers, diluents and excipients.

In a third aspect of the present invention, there is provided a method for treating a condition mediated by inappropriate activity of B-Raf kinase in a mammal in need thereof, comprising: administering to the mammal a compound of formula (I) or a pharmaceutically acceptable salt or solvate thereof.

In a fourth aspect of the present invention, there is provided a method for treating a neurotraumatic condition in a mammal in need thereof, comprising: administering to the mammal a compound of formula (I) or a pharmaceutically acceptable salt or solvate thereof.

In a fifth aspect of the present invention, there is provided a method for treating a susceptible neoplasm in a mammal in need thereof, comprising: administering to the mammal a compound of formula (I) or a pharmaceutically acceptable salt or solvate thereof.

In a sixth aspect of the present invention, there is provided a process for preparing a compound of formula (I) that includes reacting a compound of formula (IV):

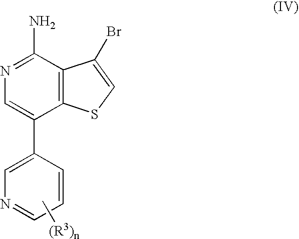

with a compound selected from formula (V), (VII) and (XII):

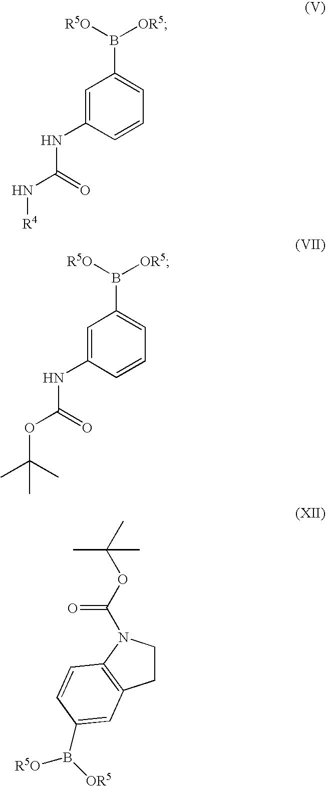

wherein in each of compound (V), (VII), and (XII), R³, R⁴ and n are as defined above, and each R⁵ is independently hydrogen or C₁ to C₆ alkyl, or the two R⁵ groups together with the boron and oxygen atoms to which they are bonded form a 5- or 6-membered ring, which is optionally substituted by 1 to 4 alkyl groups.

In a seventh aspect of the present invention, there is provided a compound of formula (I), or a pharmaceutically acceptable salt or solvate thereof for use in therapy.

In a eighth aspect of the present invention, there is provided the use of a compound of formula (I), or a pharmaceutically acceptable salt or solvate thereof in the preparation of a medicament for use in the treatment of a condition mediated by inappropriate activity of B-Raf kinase in a mammal.

In a ninth aspect of the present invention, there is provided the use of a compound of formula (I), or a pharmaceutically acceptable salt or solvate thereof in the preparation of a medicament for use in the treatment of a neurotraumatic condition in a mammal.

In a tenth aspect of the present invention, there is provided the use of a compound of formula (I), or a pharmaceutically acceptable salt or solvate thereof in the preparation of a medicament for use in the treatment of a susceptible neoplasm in a mammal.

### DETAILED DESCRIPTION OF THE INVENTION

As used herein, “a compound of the invention” means a compound of formula (I). With respect to a compound of formula (I) and any isolatable intermediates such as for example, compounds of formula (IV), (IX) and (XIV), the phrase “a compound of formula (number)” means a compound having that formula and pharmaceutically acceptable salts and solvates thereof.

As used herein, the terms “alkyl” (and “alkylene”) and similar terms refer to unsubstituted straight or branched hydrocarbon chains containing from 1 to 4 carbon atoms, unless a different number of atoms is specified. Examples of “alkyl” as used herein include, but are not limited to, methyl, ethyl, n-propyl, n-butyl, isobutyl, isopropyl, and tert-butyl. Examples of “alkylene” as used herein include, but are not limited to, methylene, ethylene, propylene, butylene, and isobutylene.

As used herein, the term “cyclohexyl” refers to an unsubstituted, saturated monocyclic carbocyclic ring.

As used herein, the term “halo” or “halogen” refers to fluoro, chloro, bromo and iodo.

As used herein, the term “haloalkyl” refers to an alkyl as defined above that is substituted one or more times with a halo as defined above.

As used herein, the term “alkoxy” refers to the group —O-alkyl, where alkyl is as defined above. Examples of “alkoxy” as used herein include, but are not limited to, methoxy, ethoxy, n-propoxy, isopropoxy, n-butoxy, and t-butoxy.

As used herein, the term “phenyl” refers to an unsubstituted phenyl unless otherwise specified.

As used herein, the term “benzyl” refers to an unsubstituted benzyl unless otherwise specified.

As used herein, the term “benzyloxy” refers to the group —O-benzyl, where benzyl is as defined above.

As used herein, the term “thienyl” refers to an unsubstituted thienyl unless otherwise specified.

As used herein, the term “isoxazolyl” refers to an unsubstituted isoxazolyl unless otherwise specified.

The present invention provides compounds of formula (I):

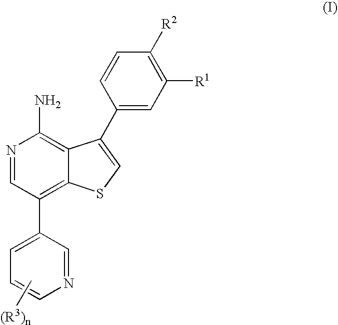

wherein:
R¹ is —N(H)—C(O)R⁴ or —N(H)—C(O)—N(H)R⁴ and R² is H; or
R¹ and R² together with the phenyl ring to which they are bonded form a moiety (i);

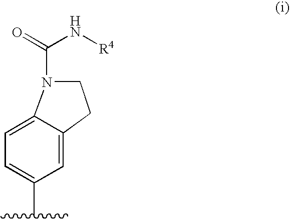

each R³ is independently selected from the group consisting of halo, alkyl, and haloalkyl;
R⁴ is selected from the group consisting of cyclohexyl; benzyl; unsubstituted, monosubstituted, or disubstituted phenyl wherein the substituent(s) is independently selected from the group consisting of halo, allyl, haloalkyl, alkoxy, haloalkoxy and benzyloxy; methylene-thienyl; and dimethyl-4-isoxazolyl; and
n is 0, 1, or 2;
   or a pharmaceutically acceptable salt or solvate thereof.

In one embodiment of the present invention, the compounds of formula (I) are defined wherein R¹ is —N(H)—C(O)R⁴. In another embodiment of the present invention, the compounds of formula (I) are defined wherein R¹ is —N(H)—C(O)—N(H)R⁴. In still another embodiment of the present invention, the compounds of formula (I) are defined wherein R¹ and R² together with the phenyl ring to which they are bonded form a moiety (i):

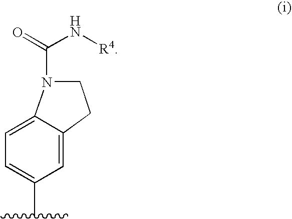

In another embodiment of the present invention, the compounds of formula (I) are defined wherein n is 0.

In another embodiment of the present invention, the compounds of formula (I) are defined wherein R⁴ is substituted phenyl. In still another embodiment of the present invention, the compounds of formula (I) are defined wherein R⁴ is a monosubstituted phenyl. In yet another embodiment of the present invention, the compounds of formula (I) are defined wherein R⁴ is monosubstituted phenyl wherein the substituent is halo, haloalkyl, alkoxy, or benzyloxy. In another embodiment of the present invention, the compounds of formula (I) are defined wherein R⁴ is disubstituted phenyl. In still another embodiment of the present invention, the compounds of formula (I) are defined wherein R⁴ is disubstituted phenyl wherein the substitutents are halo and haloalkyl. In yet another embodiment of the present invention, the compounds of formula (I) are defined wherein R⁴ is disubstituted phenyl wherein substituents are both halo. In another embodiment according to the present invention, the compounds of formula (I) are defined wherein R⁴ is benzyl. In still another embodiment according to the present invention, R⁴ is cyclohexyl.

It is to be understood that the present invention includes all combinations and subsets of the particular groups defined hereinabove.

Specific examples of compounds of the present invention include those recited in the Examples which follow and pharmaceutically acceptable salts or solvates thereof.

It will be appreciated by those skilled in the art that the compounds of the present invention may also be utilized in the form of a pharmaceutically acceptable salt or solvate thereof. The pharmaceutically acceptable salts of the compounds of formula (I) include conventional salts formed from pharmaceutically acceptable (i.e., non-toxic) inorganic or organic acids or bases as well as quaternary ammonium salts. Representative salts include the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, such as oxalic, which are not themselves pharmaceutically acceptable, may be useful in the preparation of salts useful as intermediates in obtaining compounds of this invention and these form a further aspect of the invention.

The term “solvate” as used herein refers to a complex of variable stoichiometry formed by a solute (a compound of formula (I)) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. In one embodiment the solvent used is water.

Processes for preparing pharmaceutically acceptable salts and solvates of compounds such as the compounds of formula (I) are conventional in the art. See, e.g., Burger's Medicinal Chemistry And Drug Discovery 5th Edition, Vol 1: Principles And Practice.

As will be apparent to those skilled in the art, in the processes described below for the preparation of compounds of formula (I) certain intermediates may be in the form of pharmaceutically acceptable salts or solvates of the compound. Those terms as applied to any intermediate employed in the process of preparing compounds of formula (I) have the same meanings as noted above with respect to compounds of formula (I). Processes for preparing pharmaceutically acceptable salts and solvates of intermediates are known in the art and are analogous to the process for preparing pharmaceutically acceptable salts and solvates of compounds such as the compounds of formula (I).

Certain compounds of formula (I) may exist in stereoisomeric forms (e.g. they may contain one or more asymmetric carbon atoms or may exhibit cis-trans isomerism). The individual stereoisomers (enantiomers and diastereomers) and mixtures of these are included within the scope of the present invention. The present invention also covers the individual isomers of the compounds represented by formula (I) as mixtures with isomers thereof in which one or more chiral centres are inverted. Certain compounds of formula (I) may be prepared as a mixture of regioisomers. The present invention covers both the mixture of regioisomers as well as the individual compounds. Likewise, it is understood that compounds of formula (I) may exist in tautomeric forms other than that shown in the formula and these are also included within the scope of the present invention.

As embodiments of the compounds of formula (I) are intended for use in pharmaceutical compositions it will readily be understood that in some embodiments of the present invention compounds of formula (I) are each provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

The compounds of formula (I) may be prepared singly or as compound libraries comprising at least 2, for example 5 to 1,000 compounds, and more preferably 10 to 100 compounds of formula (I). Libraries of compounds of formula (I) may be prepared by a combinatorial ‘split and mix’ approach or by multiple parallel synthesis using either solution phase or solid phase chemistry, by procedures known to those skilled in the art.

Thus according to a further aspect of the invention there is provided a compound library comprising at least 2 compounds of formula (I), or pharmaceutically acceptable salts thereof.

The compounds of the present invention are typically inhibitors of B-Raf kinase. The present invention is not limited to compounds of formula (I) which are selective for B-Raf kinase; rather, the present invention expressly contemplates compounds of formula (I) which may possess activity against kinases other than B-Raf kinase, as well. By “B-Raf inhibitor” is meant a compound which exhibits a pIC₅₀ of greater than about 5 against B-Raf kinase in the B-Raf inhibition enzyme assay described below and/or an IC₅₀ of at least about 5 against at least one cell line that overexpresses B-Raf kinase (e.g., V581E) in the cellular assay described below. In a more particular embodiment “B-Raf inhibitor” refers to a compound which exhibits a pIC₅₀ of greater than about 6.0 against B-Raf kinase in the B-Raf inhibition enzyme assay described below and/or an IC₅₀ of at least 6.0 against at least one cell line that overexpresses B-Raf kinase (e.g., V581E) in the cellular assay described below.

The present invention further provides compounds of formula (I) for use in medical therapy in a mammal, e.g. a human. In particular, the present invention provides compounds of formula (I) for use in the treatment of a condition mediated by B-Raf kinase in a mammal, and more particularly conditions mediated by inappropriate activity of B-Raf kinase in a mammal.

The inappropriate B-Raf kinase activity referred to herein is any B-Raf kinase activity that deviates from the normal B-Raf kinase activity expected in a particular mammalian subject. Inappropriate B-Raf kinase activity may take the form of, for instance, an abnormal increase in activity, or an aberration in the timing and/or control of B-Raf kinase activity. Such inappropriate activity may result then, for example, from overexpression or mutation of the protein kinase or ligand leading to inappropriate or uncontrolled activation of the receptor. Furthermore, it is also understood that unwanted B-Raf kinase activity may reside in an abnormal source, such as a malignancy. That is, the level of B-Raf activity does not have to be abnormal to be considered inappropriate, rather the activity derives from an abnormal source.

Some embodiments of the present invention provide compounds of formula (I) for use in the treatment of a neurotraumatic condition.

Other embodiments of the present invention provide compounds of formula (I) for use in the treatment of a susceptible neoplasm.

Embodiments of the present invention provide methods for the treatment of several conditions in a mammal in need thereof, all of which comprise the step of administering a compound of formula (I). Preferably, the method comprises administering to the mammal a therapeutically effective amount of a compound of formula (I). The mammal in need of treatment with a compound of the present invention is typically a human.

Some embodiments of the present invention provide methods for treating a condition mediated by B-Raf kinase in a mammal (e.g., a human), which method comprises administering to the mammal a compound of formula (I). Preferably, the method comprises administering to the mammal a therapeutically effective amount of a compound of formula (I). Conditions which are mediated by B-Raf kinase are known in the art and include but are not limited to neurotraumatic conditions and neoplasms.

Some embodiments of the present invention provide methods of treatment of a mammal suffering from a condition mediated by inappropriate activity of B-Raf kinase, which comprise administering to the mammal a compound of formula (I). Preferably, the method comprises administering to the mammal a therapeutically effective amount of a compound of formula (I). In one embodiment, the condition mediated by inappropriate activity of one or more B-Raf kinase is a neurotraumatic condition. In another embodiment, the condition mediated by inappropriate activity of B-Raf kinase is a susceptible neoplasm.

In some embodiments, the present invention provides methods for treating a neurotraumatic condition in a mammal (e.g., a human) in need thereof, which method comprises administering to the mammal a compound of formula (I). Preferably, the method comprises administering to the mammal a therapeutically effective amount of a compound of formula (I). “Neurotraumatic conditions” as defined herein include both open or penetrating head trauma, such as caused by surgery, or a closed head trauma injury, such as caused by an injury to the head region. Also included within this definition is ischemic stroke, particularly to the brain area, transient ischemic attacks following coronary by-pass and cognitive decline following other transient ischemic conditions. Ischemic stroke may be defined as a focal neurologic disorder that results from insufficient blood supply to a particular brain area, usually as a consequence of an embolus, thrombi, or local atheromatous closure of the blood vessel. Roles for stress stimuli (such as anoxia), redox injury, excessive neuronal excitatory stimulation and inflammatory cytokines in this area have been emerging and the present invention provides a means for the potential treatment of these injuries. Relatively little treatment for acute injuries such as these has been available.

In other embodiments, the present invention provides methods for treating a susceptible neoplasm (cancer or tumor) in a mammal (e.g., a human) in need thereof, which method comprises administering to the mammal a compound of formula (I). Preferably, the method comprises administering to the mammal a therapeutically effective amount of a compound of formula (I). “Susceptible neoplasm” as used herein refers to neoplasms which are susceptible to treatment with a B-Raf inhibitor. Neoplasms which have been associated with inappropriate activity of B-Raf kinase and are therefor susceptible to treatment with an B-Raf inhibitor are known in the art, and include both primary and metastatic tumors and cancers. For example, susceptible neoplasms within the scope of the present invention include but are not limited to hystocytic lymphoma, melanoma, breast cancer, small cell lung cancer, non-small cell lung cancer (e.g., lung adenocarcinoma), colon cancer, and pancreatic cancer.

As used herein, the term “treatment” refers to alleviating the specified condition, eliminating or reducing the symptoms of the condition, slowing or eliminating the progression of the condition and preventing or delaying the reoccurrence of the condition in a previously afflicted subject.

As used herein, the term “therapeutically effective amount” means an amount of a compound of formula (I) which is sufficient, in the subject to which it is administered, to elicit the biological or medical response of a cell culture, tissue, system, mammal (including human) that is being sought, for instance, by a researcher or clinician. The term also includes within its scope amounts effective to enhance normal physiological function. For example, a therapeutically effective amount of a compound of formula (I) for the treatment of a condition mediated by B-Raf kinase is an amount sufficient to treat the condition in the subject. As another example, a therapeutically effective amount of a compound of formula (I) for the treatment of a neurotraumatic condition is an amount sufficient to treat the neurotraumatic condition in the subject. Similarly, a therapeutically effective amount of a compound of formula (I) for the treatment of a susceptible neoplasm is an amount sufficient to treat the susceptible neoplasm in the subject. In one embodiment of the present invention, a therapeutically effective amount of a compound of formula (I) is an amount sufficient to regulate, modulate, bind or inhibit B-Raf kinase.

The precise therapeutically effective amount of the compounds of formula (I) will depend on a number of factors including, but not limited to, the age and weight of the subject being treated, the precise condition requiring treatment and its severity, the nature of the formulation, and the route of administration, and will ultimately be at the discretion of the attendant physician or veterinarian. Typically, the compound of formula (I) will be given for treatment in the range of 0.1 to 200 mg/kg body weight of recipient (mammal) per day and more usually in the range of 1 to 100 mg/kg body weight per day. Acceptable daily dosages, may be from about 0.1 to about 2000 mg/day, and preferably from about 0.1 to about 100 mg/day. Thus, for a 70 kg adult human being treated for a condition mediated by B-Raf kinase, the actual amount per day would usually be from 70 to 700 mg and this amount may be given in a single dose per day or more usually in a number (such as two, three, four, five or six) of sub-doses per day such that the total daily dose is the same. A therapeutically effective amount of a salt or solvate, may be determined as a proportion of the therapeutically effective amount of the compound of formula (I) per se. It is envisaged that similar dosages would be appropriate for treatment of the other conditions referred to above.

The compounds of formula (I) can be used alone in the treatment of each of the foregoing conditions or can be used to provide additive or synergistic effects with, for example, certain existing chemotherapies, and/or be used to restore effectiveness of certain existing chemotherapies and radiation.

As one aspect, the present invention provides methods of regulating, modulating, binding, or inhibiting B-Raf kinase for the treatment of conditions mediated by B-Raf kinase, by administering a therapeutically effective amount of a compound of formula (I). “Regulating, modulating, binding or inhibiting B-Raf kinase” refers to regulating, modulating, binding or inhibiting activity of B-Raf kinase, as well as regulating, modulating, binding or inhibiting overexpression of B-Raf kinase. Such conditions include neurotraumatic conditions, and certain neoplasms (including cancers and tumors) that have been associated with overexpression of B-Raf kinase.

Some embodiments of the present invention provide the use of a compound of formula (I) for the preparation of a medicament for the treatment of condition mediated by B-Raf kinase in a mammal (e.g., a human) in need thereof. In one embodiment, the present invention provides the use of a compound of formula (I) for the preparation of a medicament for the treatment of a neurotraumatic condition in a mammal. In another embodiment, the present invention provides the use of a compound of formula (I) for the preparation of a medicament for the treatment of a susceptible neoplasm in a mammal.

While it is possible that, for use in therapy, a therapeutically effective amount of a compound of formula (I) may be administered as the raw chemical, it is typically presented as the active ingredient of a pharmaceutical composition or formulation. Accordingly, the invention further provides a pharmaceutical composition comprising a compound of the formula (I). The pharmaceutical composition may further comprise one or more pharmaceutically acceptable carriers, diluents, and/or excipients. The carrier(s), diluent(s) and/or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I) with one or more pharmaceutically acceptable carriers, diluents and/or excipients.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula (I), depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I) can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula (I) may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use.

Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

Compounds of formula (I) may be prepared using the processes described below. In all of the schemes described below, it is understood that protecting groups may be employed where necessary in accordance with general principles known to those of skill in the art, for example, see T. W. Green and P. G. M. Wuts (1991) Protecting Groups in Organic Synthesis, John Wiley & Sons. These groups may be removed at a convenient stage of the compound synthesis using methods known to those of skill in the art. The selection of processes as well as the reaction conditions and order of their execution shall be consistent with the preparation of compounds of formula (I).

Compounds of formula (I) may be conveniently prepared by the processes outlined in Schemes 1-3 below. In each of Schemes 1-3 and the General Intermediate Scheme, all variables are as defined above. Each of Schemes 1-3 utilize an intermediate (IV) prepared by the following General Intermediate Scheme:

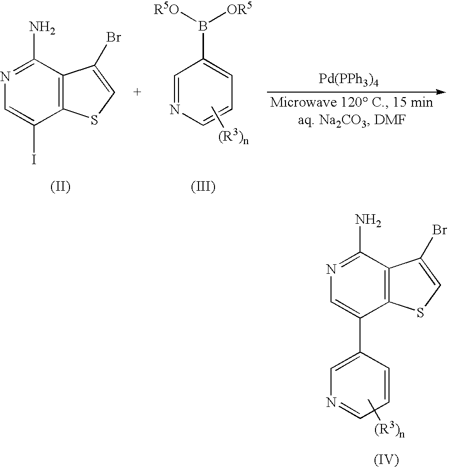

wherein R³ and n are as defined above with respect to Formula (I), and R⁵ is independently hydrogen or C₁ to C₆ alkyl, or the two R⁵ groups together with the boron and oxygen atoms to which they are bonded form a 5- or 6-membered ring, which is optionally substituted by 1 to 4 alkyl groups.

Compound (II), which can be prepared according to the reported method in WO 2004100947, and compound (III), which is commercially available or can be readily prepared utilizing commercially available compounds by those of ordinary skill in the art, in a 1:1.1 molar ratio are dissolved in DMF and 2M aqueous Na₂CO₃, which is typically used in a 5:1 volume ratio. As will be understood by those skilled in the art, other molar ratios of compounds (II) and (III) such as 1:1 to 1:1.5 could be used. Also, 2M Na₂CO₃ could be used from 2 to 10 equivalence with respect to compound (III). In place of Na₂CO₃, other bases such as K₂CO₃, K₃PO₄, Cs₂CO₃, CsF, Ba(OH)₂, NaOH, NaHCO₃ could be used. Instead of or in addition to DMF, other solvent systems such as DME, Dioxane, THF, Toluene, Xylene, MeOH, ethanol, H₂O, MeCN, NMP or a mixture of two or more of them could be used. The catalyst is preferably Pd(PPh₃)₄; however, it is understood that other catalysts such as Pd(OAc)₂, Pd₂(dba)₃, [PdCl(allyl)]₂, with a suitable ligand such as PPh₃, PCy₃, (t-Bu)₂POH, (t-Bu)₃P could be used. Also, phosphine-free palladium such as Pd/C, and polymer bound palladium may be used. The use of a different catalyst may alter the time, temperature, and/or solvent to be used as will be understood by one skilled in the art. The reaction is preferably performed using microwave heating at 120° C. for 15 minutes. However, other modes of heating such as oil baths or hot plates may also be used. Also, other temperatures of 60 to 180° C. and times of 5 min to 24 h may be utilized, with the general understanding that higher reaction temperatures typically will require shorter reaction times. The mixture can be filtered and washed with DMF. The filtrate can be purified by SCX cartridge via capture-and-release, for example. The crude can be recrystallized from MeOH and a small amount of CH₂Cl₂, for example, to give compound IV.

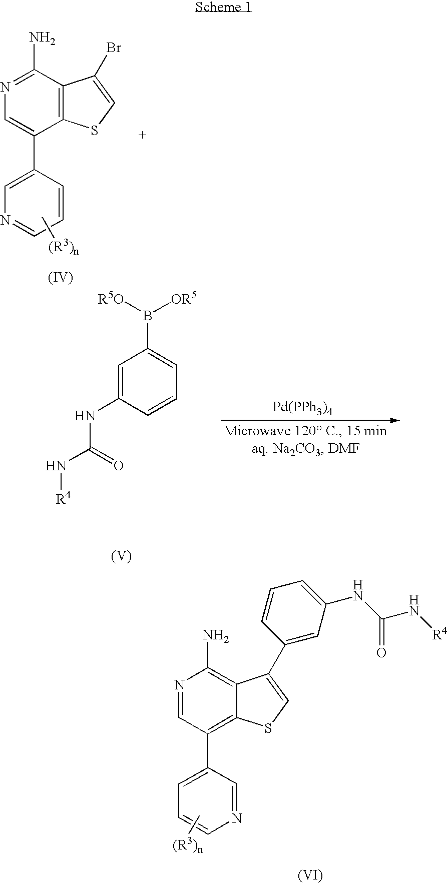

wherein R³, R⁴, and n are as defined above with respect to Formula (I), and R⁵ is independently hydrogen or C₁ to C₆ alkyl, or the two R⁵ groups together with the boron and oxygen atoms form a 5- or 6-membered ring, which is optionally substituted by 1 to 4 alkyl groups.

Compound (IV) and compound (V), which is commercially available or can be readily prepared utilizing commercially available compounds by those of ordinary skill in the art, in a molar ratio that is typically 1:1.3, but can vary from 1:1 to 1:3 are dissolved in DME and 2M aqueous Na₂CO₃, which is typically used in a 4:1 volume ratio. 2M Na₂CO₃ could be used from 2 to 10 equivalence with respect to compound (V). In place of Na₂CO₃, other bases such as K₂CO₃, K₃PO₄, Cs₂CO₃, CsF, Ba(OH)₂, NaOH, NaHCO₃ could be used. Instead of or in addition to DME, other solvent systems such as DMF, dioxane, THF, toluene, xylene, MeOH, ethanol, H₂O, MeCN, NMP or a mixture of two or more of them could be used. The catalyst is preferably Pd(PPh₃)₄; however, it is understood that other catalysts such as Pd(OAc)₂, Pd₂(dba)₃, [PdCl(allyl)]₂, with a suitable ligand such as PPh₃, PCy₃, (t-Bu)₂POH, (t-Bu)₃P could be used. Also, phosphine-free palladium such as Pd/C, and polymer bound palladium may be used. The use of a different catalyst may alter the time, temperature, and/or solvent to be used as will be understood by one skilled in the art. The reaction is preferably performed using microwave heating at 120° C. for 15 minutes. However, other modes of heating such as oil baths or hot plates may also be used. Also, other temperatures of 60 to 180° C. and times of 5 min to 24 h may be utilized, with the general understanding that higher reaction temperatures typically will require shorter reaction times. Compound (VI) can be obtained by concentration in vacuo and purification by mass directed LC/MS, for example.

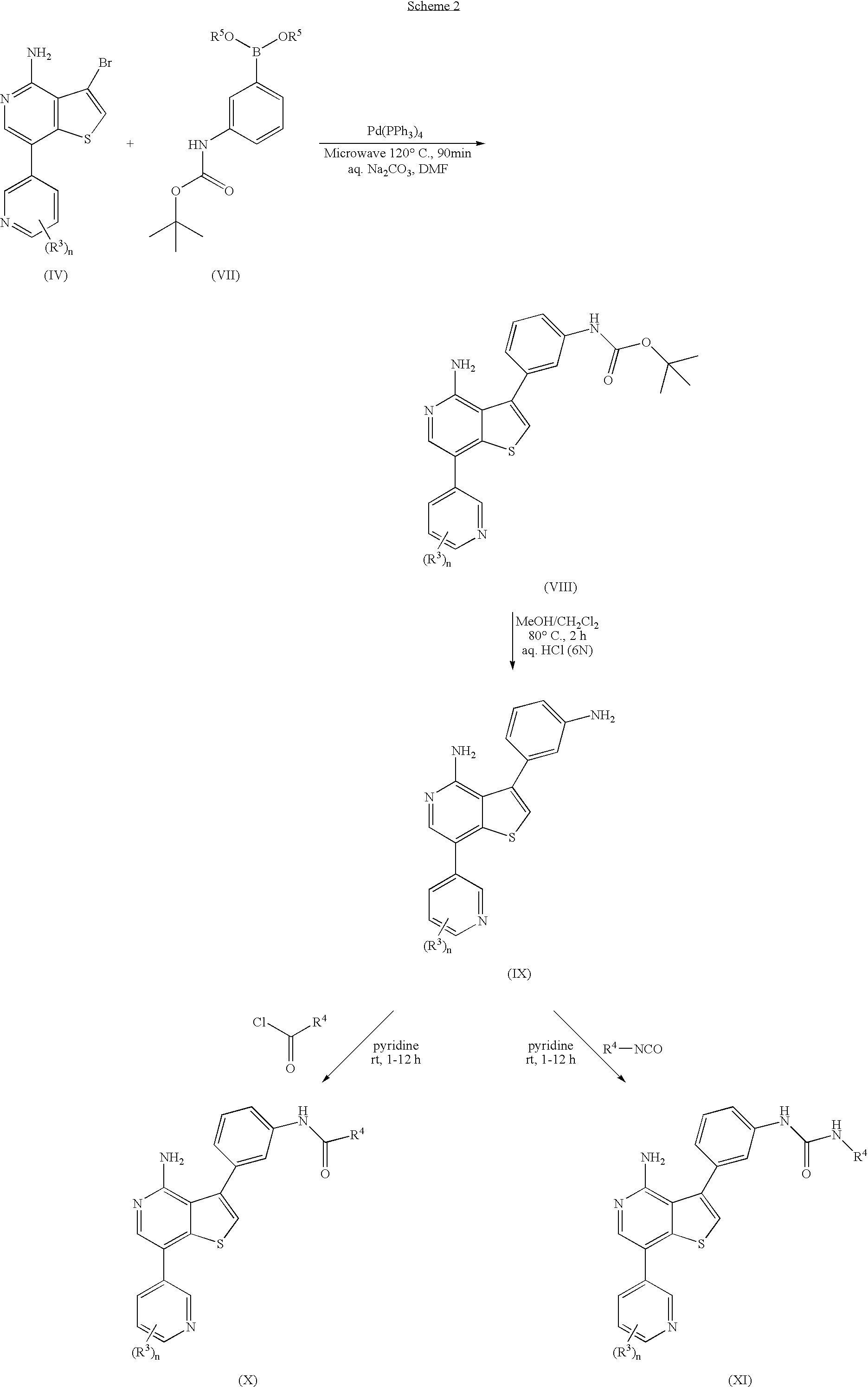

wherein R³, R⁴, and n are as defined above with respect to Formula (I), and R⁵ is independently hydrogen or C₁ to C₆ alkyl, or the two R⁵ groups together with the boron and oxygen atoms form a 5- or 6-membered ring, which is optionally substituted by 1 to 4 alkyl groups.

To prepare compound (VIII), compound (IV) and compound (VII), which is commercially available or can be readily prepared utilizing commercially available compounds by those of ordinary skill in the art, in a molar ratio that is typically 1:1.2, but can vary from 1:1 to 1:3 are dissolved in DME and 2M aqueous Na₂CO₃, which is typically used in a 4:1 volume ratio. 2M Na₂CO₃ could be used from 2 to 10 equivalence with respect to compound (VII). In place of Na₂CO₃, other bases such as K₂CO₃, K₃PO₄, Cs₂CO₃, CsF, Ba(OH)₂, NaOH, NaHCO₃ could be used. Instead of or in addition to DME, other solvent systems such as DMF, dioxane, THF, toluene, xylene, MeOH, ethanol, H₂O, MeCN, NMP or a mixture of two or more of them could be used. The catalyst is preferably Pd(PPh₃)₄; however, it is understood that other catalysts such as Pd(OAc)₂, Pd₂(dba)₃, [PdCl(allyl)]₂, with a suitable ligand such as PPh₃, PCy₃, (t-Bu)₂POH, (t-Bu)₃P could be used. Also, phosphine-free palladium such as Pd/C, and polymer bound palladium may be used. The use of a different catalyst may alter the time, temperature, and/or solvent to be used as will be understood by one skilled in the art. The reaction is preferably performed using microwave heating at 120° C. for 90 minutes. However, other modes of heating such as oil baths or hot plates may also be used. Also, other temperatures of 60 to 180° C. and times of 5 min to 24 h may be utilized, with the general understanding that higher reaction temperatures typically will require shorter reaction times. The filtrate can be purified by SCX cartridge via capture-and-release, for example. Concentration in vacuo can be used to provide compound (VIII).

To prepare compound (IX), a solution of compound (VIII) in 1:1 MeOH:CH₂Cl₂ is added to 6N aqueous HCl, with the MeOH:CH₂Cl₂ and 6N HCl in a 10:1 volume ratio. As will be understood by those skilled in the art, other solvent systems such as ethanol, dioxane, H₂O, CHCl₃, CH₂ClCH₂Cl, and other volume ratios such as 10:1 to 1:10 can be used. The reaction mixture is heated at 80° C. for 2 h. As will be understood to those skilled in the art, the temperature and time can be various temperatures and times, such as rt to 120° C. and 1 to 12 hours. The reaction mixture is diluted with aqueous Na₂CO₃ and extracted with CH₂Cl₂. Other elution solvents, such as NaHCO₃, K₂CO₃, NaOH, KHCO₃, and other extraction solvents, such as CHCl₃, CH₂ClCH₂Cl, EtOAc can also be used. The filtrate can be purified by, for example, SCX cartridge via capture-and-release, and concentrated in vacuo.

To prepare compound (X), R⁴C(O)Cl is added to a solution of compound (IX) in pyridine, with the molar ratio of compound (IX) to compound R⁴C(O)Cl being from 1:1 to 1:1.5, preferably 1:1.2. While R⁴C(O)Cl is a preferred reactant, R⁴C(O)Br could also be used in similar ratios. While the preferred solvent is pyridine, those skilled in the art will understand that other solvents, such as CH₂Cl₂, CHCl₃, CH₂ClCH₂Cl with a suitable amine such as Et₃N, pyridine, N,N-diethyl-2-propanamine can be used. The reaction mixture is stirred at room temperature for 2 hours. As will be understood by those skilled in the art, various temperatures such as 0 to 60° C. and times such as 1 to 12 hours can be used. Compound (X) can be obtained by concentration in vacuo and purification by mass directed LC/MS, for example.

To prepare compound (XI), R—NCO is added to a solution of compound (IX) in pyridine, with the molar ratio of compound (TX) to compound R—NCO being from 1:1 to 1:1.5, preferably 1:1.2. While the preferred solvent is pyridine, those skilled in the art will understand that other solvents, such as CH₂Cl₂, CHCl₃, CH₂ClCH₂Cl, can be used. The reaction mixture is stirred at room temperature for 2 hours. As will be understood by those skilled in the art, various temperatures such as 0 to 60° C. and times such as 1 to 12 hours can be used. Compound (XI) can be obtained by concentration in vacuo and purification by mass directed LC/MS, for example.

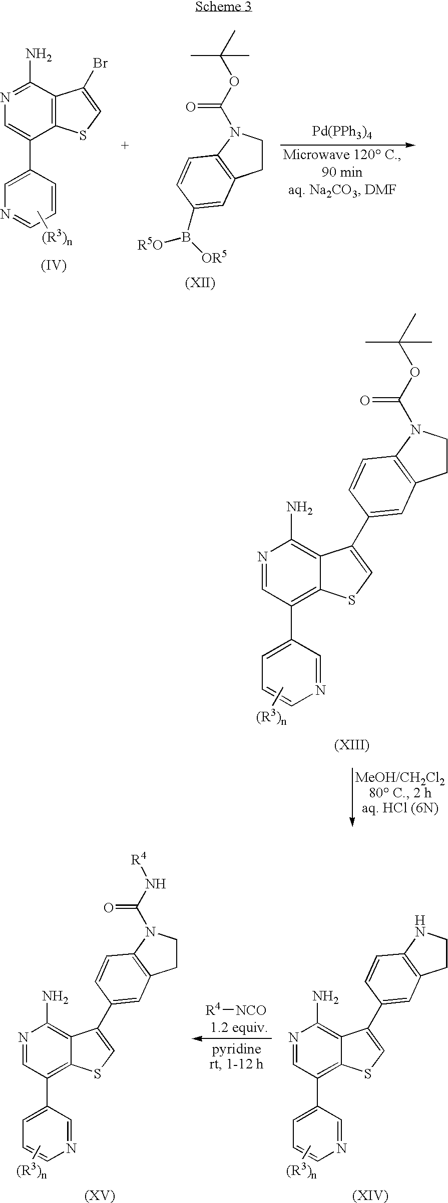

wherein R³, R⁴, and n are as defined above with respect to Formula (I), and R⁵ is independently hydrogen or C₁ to C₆ allyl, or the two R⁵ groups together with the boron and oxygen atoms form a 5- or 6-membered ring, which is optionally substituted by 1 to 4 alkyl groups.

To prepare compound (XIII), compound (IV) and compound (XII), which is commercially available or can be readily prepared utilizing commercially available compounds by those of ordinary skill in the art, in a molar ratio that is typically 1:1.2, but can vary from 1:1 to 1:3 are dissolved in DME and 2M aqueous Na₂CO₃, which is typically used in a 4:1 volume ratio. 2M Na₂CO₃ could be used from 2 to 10 equivalence with respect to compound (XII). In place of Na₂CO₃, other bases such as K₂CO₃, K₃PO₄, Cs₂CO₃, CsF, Ba(OH)₂, NaOH, NaHCO₃ could be used. Instead of or in addition to DME, other solvent systems such as DMF, dioxane, THF, toluene, xylene, MeOH, ethanol, H₂O, MeCN, NMP or a mixture of two or more of them could be used. The catalyst is preferably Pd(PPh₃)₄; however, it is understood that other catalysts such as Pd(OAc)₂, Pd₂(dba)₃, [PdCl(allyl)]₂, with a suitable ligand such as PPh₃, PCy₃, (t-Bu)₂POH, (t-Bu)₃P could be used. Also, phosphine-free palladium such as Pd/C, and polymer bound palladium may be used. The use of a different catalyst may alter the time, temperature, and/or solvent to be used as will be understood by one skilled in the art. The reaction is preferably performed using microwave heating at 120° C. for 90 minutes. However, other modes of heating such as oil baths or hot plates may also be used. Also, other temperatures of 60 to 180° C. and times of 5 min to 24 h may be utilized, with the general understanding that higher reaction temperatures typically will require shorter reaction times. The filtrate can be purified by SCX cartridge via capture-and-release, for example. Concentration in vacuo can be used to provide compound (XIII).

To prepare compound (XIV), a solution of compound (XIII) in 1:1 MeOH:CH₂Cl₂ is added to 6N aqueous HCl, with the MeOH:CH₂Cl₂ and 6N HCl in a 10:1 volume ratio. As will be understood by those skilled in the art, other solvent systems such as ethanol, dioxane, H₂O, CHCl₃, CH₂ClCH₂Cl, and other volume ratios such as 10:1 to 1:10 can be used. The reaction mixture is heated at 80° C. for 2 h. As will be understood to those skilled in the art, the temperature and time can be various temperatures and times, such as rt to 120° C. and 1 to 12 hours. The reaction mixture is diluted with aqueous Na₂CO₃ and extracted with CH₂Cl₂. Other elution solvents, such as NaHCO₃, K₂CO₃, NaOH, KHCO₃, and other extraction solvents, such as CHCl₃, CH₂ClCH₂Cl, EtOAc can also be used. The filtrate can be purified by, for example, SCX cartridge via capture-and-release, and concentrated in vacuo.

To prepare compound (XV), R⁴—NCO is added to a solution of compound (XV) in pyridine, with the molar ratio of compound (XIV) to compound R⁴—NCO being from 1:1 to 1:1.5, preferably 1:1.2. While the preferred solvent is pyridine, those skilled in the art will understand that other solvents, such as CH₁₂Cl₂, CHCl₃, CH₂ClCH₂Cl with a suitable amine such as Et₃N, pyridine, N,N-diethyl-2-propanamine can be used. The reaction mixture is stirred at room temperature for 2 hours. As will be understood by those skilled in the art, various temperatures such as 0 to 60° C. and times such as 1 to 12 hours can be used. Compound (XV) can be obtained by concentration in vacuo and purification by mass directed LC/MS, for example.

Based upon this disclosure and the examples contained herein one skilled in the art can readily convert a compound of formula (I) or a pharmaceutically acceptable salt or solvate thereof into a different compound of formula (I), or a pharmaceutically acceptable salt or solvate thereof.

The following examples are intended for illustration only and are not intended to limit the scope of the invention in any way, the invention being defined by the claims which follow.

### EXAMPLES

As used herein, the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
g (grams);
L (liters);
μL (microliters);
M (molar);
i. v. (intravenous);
MHz (megahertz);
mmol (millimoles);
min (minutes);
mp (melting point);
Tᵣ (retention time);
MeOH (methanol);
mg (milligrams);
mL (milliliters);
psi (pounds per square inch);
mM (millimolar);
Hz (Hertz);
mol (moles);
rt (room temperature);
h (hours);
TLC (thin layer chromatography);
RP (reverse phase);
i-PrOH (isopropanol);
TEA (triethylamine);
TFAA (trifluoroacetic anhydride);
DMSO (dimethylsulfoxide);
DME (1,2-dimethoxyethane);
DCE (dichloroethane);
DMPU (N,N′-dimethylpropyleneurea);
IBCF (isobutyl CHCl₃ate);
HOSu (N-hydroxysuccinimide);
mCPBA (meta-chloroperbenzoic acid;
BOC (tert-butyloxycarbonyl);
DCC (dicyclohexylcarbodiimide);
Ac (acetyl);
TMSE (2-(trimethylsilyl)ethyl);
TTPS (triisopropylsilyl);
DMAP (4-dimethylaminopyridine);
ATP (adenosine triphosphate);
DMEM (Dulbecco's modified Eagle medium);
HPLC (high pressure liquid chromatography);
BOP (bis(2-oxo-3-oxazolidinyl)phosphinic chloride);
TBAF (tetra-n-butylammonium fluoride);
HBTU (O-Benzotriazole-1-yl-N,N,N′,N′-tetramethyluronium hexafluorophosphate);
HEPES (4-(2-hydroxyethyl)-1-piperazine ethane sulfonic acid);
DPPA (diphenylphosphoryl azide);
fHNO3 (fumed HNO₃);
EDC (ethylcarbodiimide hydrochloride); and
EDTA (ethylenediaminetetraacetic acid).
TFA (trifluoroacetic acid);
THF (tetrahydrofuran);
AcOEt (EtOAc);
DCM (CH2Cl2);
DMF (N, N-dimethylformamide);
(CDI (1,1-carbonyldiimidazole);
HOAc (acetic acid);
HOBt (1-hydroxybenzotraizole);
FMOC (9-fluorenylmethoxycarbonyl);
CBZ (benzyloxycarbonyl);
atm (atmosphere);
TMS (trimethylsilyl);
TBS (t-butyldimethylsilyl);
BSA (bovine serum albumin);
HRP (horseradish peroxidase);
   All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded on a Varian VXR-300, a Varian Unity-300, a Varian Unity-400 instrument, a Brucker AVANCE-400, or a General Electric QE-300.

Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

Low-resolution mass spectra (MS) were recorded on a Waters micromass ZQ2000; 2695 Alliance; 2996 Photodiode Array Detector. Preparative LC/MS purification uses the following condition:

Waters FractionLynx LC/MS condition
Autosampler/Fractioncollector: 2767 inject collector
Waste collector: waters fraction collector 2
HPLC: 2525 pump
Detector: 2996 Photodiode Array Detector
MS: ZQ2000
Make up pump: waters reagent manager
   Purification protocol
Loading: 5-100 mg,
4 gradient methods, Cycle time: 15 min
Flow rate: 40 mL/min
Column: XTerra™ MSC₁₈, 30×150 mm (10 μmm)
Injection Volume: 1800 μL
Temperature: rt
   Basic condition mobile phase
A—(Pure H₂O: 3 L+28% Ammonia solution 11 mL)
B—100% Acetonitrile
   Acidic condition mobile phase
A—(Pure H₂O: 3 L+100% Formic acid 3 mL)
B—(Acetonitrile: 3 L+100% Formic acid 3 mL)
   Make up solvent
   20% H₂O+80% Methanol+10 mM ammonium acetate
   Gradient: 6 gradient methods for purification (Solvent B ratio)

| | | Method | Method | Method | Method | Method | Method |
|---|---|---|---|---|---|---|---|
| Time | flow | 1 | 2 | 3 | 4 | 5 | 6 |
| 0 | 1 | 5 | 5 | 5 | 5 | 5 | 5 |
| 0.5 | 40 | 5 | 5 | 5 | 5 | 5 | 5 |
| 1 | 40 | 5 | 10 | 20 | 35 | 50 | 65 |
| 10 | 40 | 25 | 25 | 40 | 55 | 75 | 90 |
| 13.5 | 40 | 100 | 100 | 100 | 100 | 100 | 100 |
| 15 | 40 | 100 | 100 | 100 | 100 | 100 | 100 |

All mass spectra were taken under electrospray ionization (ESI), chemical ionization (CI), and electron impact (EI) or by fast atom bombardment (FAB) methods. All reactions were monitored by thin-layer chromatography on 0.25 mm E. Merck silica gel plates (60F-254), visualized with UV light, 5% ethanolic phosphomolybdic acid or p-anisaldehyde solution or mass spectrometry (electrospray or AP). Flash column chromatography was performed on silica gel (230-400 mesh, Merck) or using automated silica gel chromatography (Yamazen Fast Flow Liquid Chromatography, UV detection triggering sample collection).

Microwave irradiation was performed on a Personal Chemistry Smithsynthesizer or Creator.

SCX purification: Varian Mega Bond Elut SCX; General procedure: A SCX cartridge was rinsed with MeOH, and then crude mixture was dissolved into a suitable solvent such as MeOH, DCM etc. and loaded on the cartridge. And then the cartridge was rinsed with methanol and dichloromethane successively. The product was isolated by elution with a 2M ammonia solution in methanol (for some cases, mixed with DCM), followed by concentration in vacuo.

General intermediate: 3-Bromo-7-(3-pyridinyl)thieno[3,2-c]pyridin-4-amine

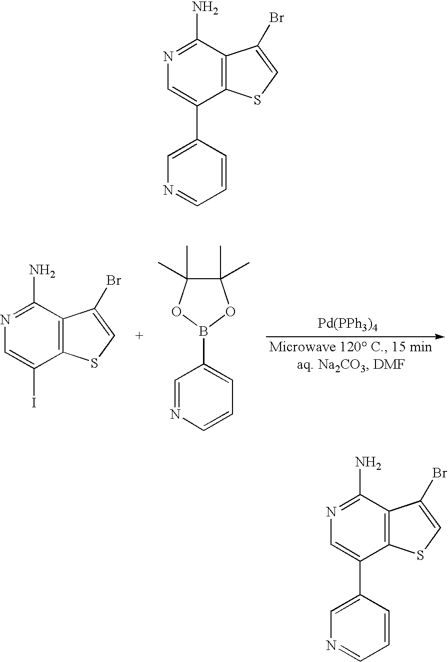

3-Bromo-7-iodothieno[3,2-c]pyridin-4-amine (0.71 g, 2 nmol), which was prepared according to the reported method in WO 2004100947, and 3-(4,4,5,5-tetramethyl-1,3,2-dioxaborolan-2-yl)pyridine (0.36 g, 2.2 mmol, commercial available) were dissolved in DMF (10 mL) and aqueous Na₂CO₃ (2M, 2 mL). The resulting solution and Pd(PPh₃)₄ (10 mol %) were added to a Microwave vial (10-20 mL). After capping, the mixture was heated with Creator at 120° C. for 15 min. The mixture was filtered and washed with DMF. The filtrate was purified by SCX cartridge via capture-and-release. The crude was recrystallized from MeOH and a small amount of CH₂Cl₂ to give the product (0.42 g, 69%) as solid. ¹H NMR (400 MHz, d₆-DMSO) ppm 8.81 (dd, J=0.8, 2.3 Hz, 1H), 8.61 (dd, J=1.8, 4.8 Hz, 1H), 8.04 (ddd, J=0.8, 2.3, 7.8 Hz, 1H), 7.97 (s, 1H), 7.87 (s, 1H), 7.52-7.56 (m, 1H), 6.75 (s, 2H). MS (ESI) m/z=306, 308 [M+H]+.

### Example 1

### N-[3-(4-Amino-7-pyridin-3-ylthieno[3,2-c]pyridin-3-yl)phenyl]-N′-[2-fluoro-5-(trifluoromethyl)phenyl]urea

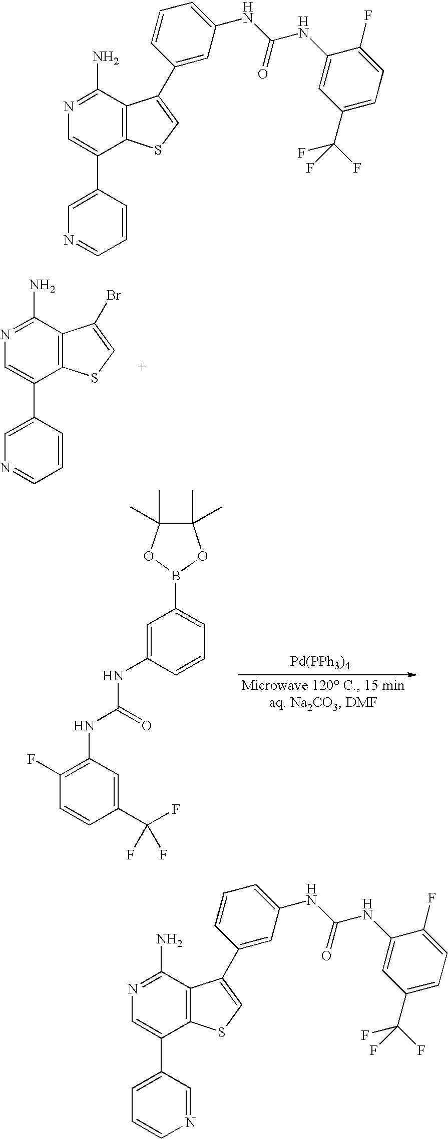

3-Bromo-7-(3-pyridinyl)thieno[3,2-c]pyridin-4-amine (30.6 mg, 0.1 mmol) and AT-[2-fluoro-5-(trifluoromethyl)phenyl]-N′-[3-(4,4,5,5-tetramethyl-1,3,2-dioxaborolan-2-yl)phenyl]urea (55 mg, 0.13 mmol) were dissolved in DME (1 mL) and aqueous Na₂CO₃ (2M, 0.25 mL). The resulting solution and Pd(PPh₃)₄ (10 mol %) were added to a Microwave vial (2-5 mL). After capping, the mixture was heated with Creator at 120° C. for 15 min. The mixture was filtered and washed with MeOH/CH₂Cl₂. The filtrate was purified by SCX cartridge via capture-and-release. Concentration in vacuo and purification by mass directed LC/MS gave the title compound (6.8 mg, 13%). ¹H NMR (400 MHz, d₆-DMSO) ppm 9.57 (s, 1H), 9.14 (d, J=2.3 Hz, 1H), 8.89 (d, J=2.0 Hz, 1H), 8.63 (dd, J=1.5, 4.8 Hz, 1H), 8.58 (dd, J=2.0, 7.1 Hz, 1H), 8.11 (dt, J=2.0, 7.8 Hz, 1H), 7.98 (s, 1H), 7.71 (t, J=1.8 Hz, 1H), 7.37-7.59 (m, 6H), 7.14 (d, J=7.1 Hz, 1H), 5.68 (s, 2H). MS (ESI) m/z=524 [M+H]+.

### Example 2

### N-[3-(4-Amino-7-pyridin-3-ylthieno[3,2-c]pyridin-3-yl)phenyl]-3-(trifluoromethyl)benzamide

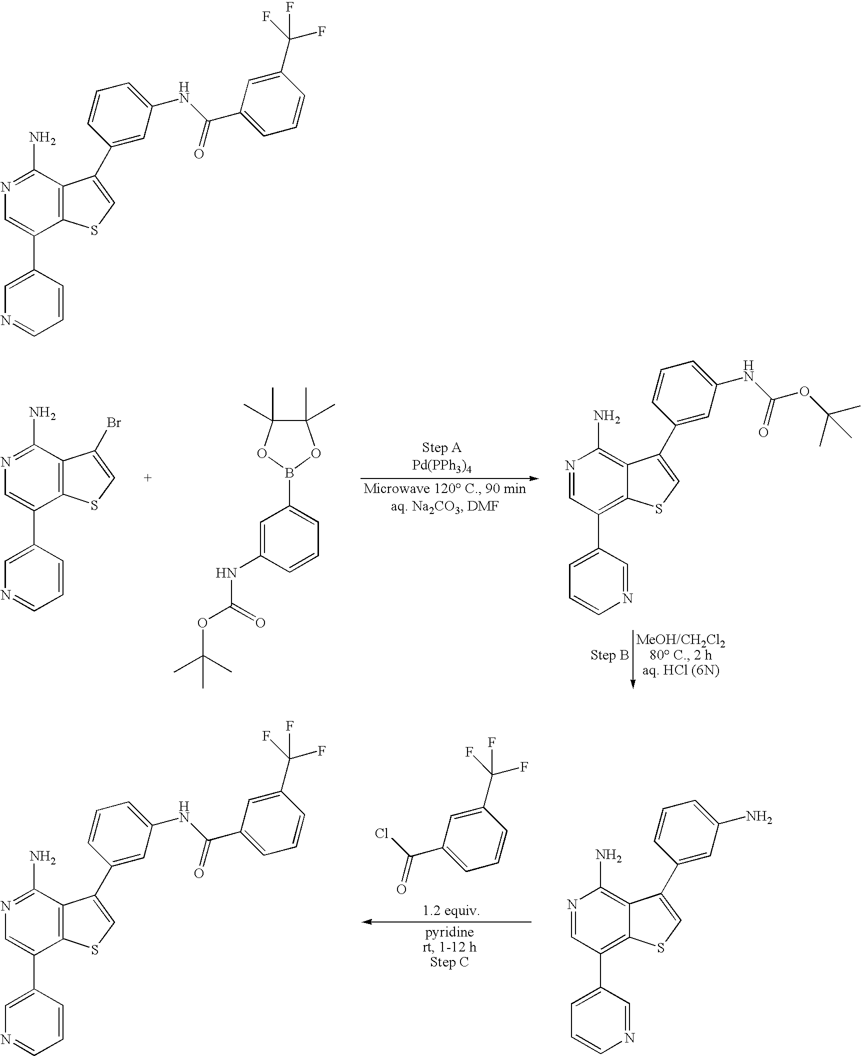

### Steps A and B: 3-(3-Aminophenyl)-7-(3-pyridinyl)thieno[3,2-c]pyridin-4-amine

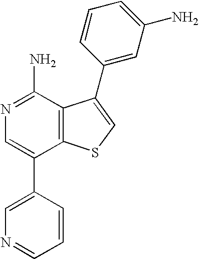

3-Bromo-7-(3-pyridinyl)thieno[3,2-c]pyridin-4-amine (417 mg, 1.36 mmol) and 1,1-dimethylethyl[3-(4,4,5,5-tetramethyl-1,3,2-dioxaborolan-2-yl)phenyl]carbamate (522 mg, 1.63 mmol) were dissolved in DME (10 mL) and aqueous Na₂CO₃ (2M, 2.5 mL). The resulting solution and Pd(PPh₃)₄ (10 mol %) were added to a Microwave vial (10-20 mL). After capping, the mixture was heated with Creator at 120° C. for 90 min. The mixture was filtered and washed with MeOH/CH₂Cl₂. The filtrate was purified by SCX cartridge via capture-and-release. Concentration in vacuo gave the product utilized in the next step without characterization.

To a solution of the above residue in 1:1 MeOH:CH₂Cl₂ (20 mL) was added aqueous HCl (6N, 2 mL). The reaction mixture was heated by oil bath at 80° C. for 2 h. The reaction mixture was diluted with aqueous Na₂CO₃ and extracted with CH₂Cl₂. The filtrate was purified by SCX cartridge via capture-and-release. Concentration in vacuo gave the compound produced by step B (300 mg, 70%). ¹H NMR (400 MHz, d₆-DMSO) ppm 8.87 (dd, J=0.8, 2.3 Hz, 1H), 8.61 (dd, J=1.8, 4.8 Hz, 1H), 8.09 (ddd, J=0.8, 2.3, 7.8 Hz, 1H), 7.94 (s, 1H), 7.53-7.57 (m, 1H), 7.47 (s, 1H), 7.17 (t, J=7.8 Hz, 1H), 6.54-6.69 (m, 3H), 5.77 (s, 2H), 5.38 (s, 2H). MS (ESI) m/z=319 [M+H]+.

### Step C: N-[3-(4-Amino-7-pyridin-3-ylthieno[3,2-c]pyridin-3-yl)phenyl]-3-(trifluoromethyl)benzamide (title compound)

To a solution of the above residue (15 mg, 0.047 mmol) in pyridine (1 mL) was added 3-(trifluoromethyl)benzoyl chloride (1.2 equiv., 0.0565 mmol). The reaction mixture was stirred at rt for 2 h. Concentration in vacuo and purification by mass directed LC/MS gave the title compound without the calculation of the yield. ¹H NMR (400 MHz, d₆-DMSO) ppm 10.67 (s, 1H), 8.89 (dd, J=0.8, 2.3 Hz, 1H), 8.62 (dd, J=1.8, 4.8 Hz, 1H), 8.31 (s, 1H), 8.28 (d, J=7.8 Hz, 1H), 8.10-8.13 (m, 1H), 7.92-8.00 (m, 4H), 7.81 (t, J=7.8 Hz, 1H), 7.61 (s, 1H), 7.54-7.58 (m, 2H), 7.27 (dt, J=1.2, 7.8 Hz, 1H), 5.68 (s, 2H). MS (ESI) m/z=491 [M+H]+.

### Example 3

### N-[3-(4-Amino-7-pyridin-3-ylthieno[3,2-c]pyridin-3-yl)phenyl]-2-phenylacetamide

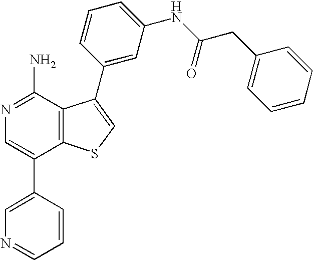

A procedure similar to Example 2 was used, with phenylacetyl chloride being substituted for 3-(trifluoromethyl)benzoyl chloride, to prepare the title compound.

¹H NMR (400 MHz, d₆-DMSO) ppm 10.39 (s, 1H), 8.87 (dd, J=0.8, 2.3 Hz, 1H), 8.62 (dd, J=1.5, 4.8 Hz, 1H), 8.10 (ddd, J=0.8, 1.8, 8.1 Hz, 1H), 7.97 (s, 1H), 7.78 (t, J=1.8 Hz, 1H), 7.71 (ddd, J=0.8, 2.0, 8.1 Hz, 1H), 7.53-7.57 (m, 2H), 7.48 (t, J=8.1 Hz, 1H), 7.15-7.36 (m, 6H), 5.63 (s, 2H), 3.67 (s, 2H). MS (EST) m/z=437 [M+H]+.

### Example 4

### N-[3-(4-Amino-7-pyridin-3-ylthieno[3,2-c]pyridin-3-yl)phenyl]-2-(2-thienyl)acetamide

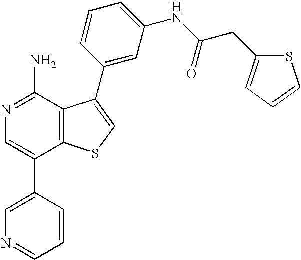

A procedure similar to Example 2 was used, with 2-thienylacetyl chloride being substituted for 3-(trifluoromethyl)benzoyl chloride, to prepare the title compound.

¹H NMR (400 MHz, d₆-DMSO) ppm 10.43 (s, 1H), 8.88 (dd, J=0.8, 2.2 Hz, 1H), 8.62 (dd, J=1.5, 4.8 Hz, 1H), 8.10 (ddd, J=0.8, 2.2, 7.8 Hz, 1H), 7.97 (s, 1H), 7.78 (t, J=1.8 Hz, 1H), 7.68-7.71 (m, 1H), 7.54-7.57 (m, 2H), 7.49 (t, J=8.1 Hz, 1H), 7.40 (dd, J=1.5, 5.0 Hz, 1H), 7.18 (dt, J=1.5, 7.8 Hz, 1H), 6.96-7.01 (m, 2H), 5.63 (s, 2H), 3.91 (s, 2H). MS (ESI) m/z=443 [M+H]+.

### Example 5

### N-[3-(4-Amino-7-pyridin-3-ylthieno[3,2-c]pyridin-3-yl)phenyl]-2,5-difluorobenzamide

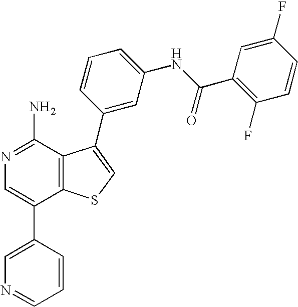

A procedure similar to Example 2 was used, with 2,5-difluorobenzoyl chloride being substituted for 3-(trifluoromethyl)benzoyl chloride, to prepare the title compound.

¹H NMR (400 MHz, d₆-DMSO) ppm 10.72 (s, 1H), 8.88 (dd, J=0.8, 2.3 Hz, 1H), 8.62 (dd, J=1.5, 4.8 Hz, 1H), 8.11 (ddd, J=0.8, 2.3, 7.8 Hz, 1H), 7.98 (s, 1H), 7.90 (t, J=1.8 Hz, 1H), 7.81-7.84 (m, 1H), 7.43-7.60 (m, 6H), 7.26 (dt, J=1.5, 8.1 Hz, 1H), 5.66 (s, 2H). MS (ESI) m/z=459 [M+H]+.

### Example 6

### N-[3-(4-Amino-7-pyridin-3-ylthieno[3,2-c]pyridin-3-yl)phenyl]benzamide

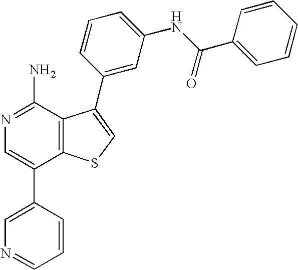

A procedure similar to Example 2 was used, with benzoyl chloride being substituted for 3-(trifluoromethyl)benzoyl chloride, to prepare the title compound.

¹H NMR (400 MHz, d₆-DMSO) ppm 10.46 (s, 1H), 8.89 (dd, J=0.8, 2.3 Hz, 1H), 8.62 (dd, J=1.5, 4.8 Hz, 1H), 8.11 (ddd, J=0.8, 2.3, 8.1 Hz, 1H), 7.91-7.99 (m, 5H), 7.52-7.63 (m, 6H), 7.23 (dt, J=1.5, 8.1 Hz, 1H), 5.68 (s, 2H). MS (ESI) m/z=423 [M+H]+.

### Example 7

### N-{3-[4-Amino-7-(3-pyridinyl)thieno[3,2-c]pyridin-3-yl]phenyl}cyclohexanecarboxamide

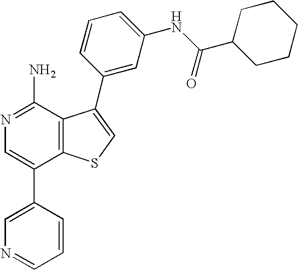

A procedure similar to Example 2 was used, with cyclohexanecarbonyl chloride being substituted for 3-(trifluoromethyl)benzoyl chloride, to prepare the title compound.

¹H NMR (400 MHz, d₆-DMSO) ppm 10.02 (s, 1H), 8.88 (dd, J=0.8, 2.3 Hz, 1H), 8.62 (dd, J=1.8, 4.8 Hz, 1H), 8.11 (ddd, J=0.8, 2.3, 7.8 Hz, 1H), 7.97 (s, 1H), 7.80 (t, J=1.8 Hz, 1H), 7.68-7.72 (m, 1H), 7.54-7.58 (m, 2H), 7.46 (t=8.1 Hz, 1H), 7.13 (dt, J=1.5, 7.8 Hz, 1H), 5.64 (s, 2H), 2.31-2.38 (m, 1H), 1.16-1.83 (m, 10H). MS (ESI) m/z=429 [M+H]+.

### Example 8

### N-[3-(4-Amino-7-pyridin-3-ylthieno[3,2-c]pyridin-3-yl)phenyl]-N′-(3-methoxyphenyl)urea

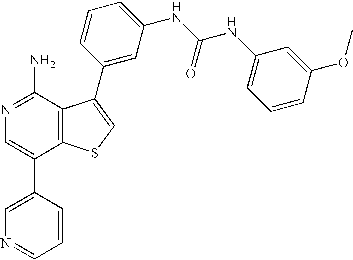

A procedure similar to Example 2 was used, with 1-isocyanato-3-(methyloxy)benzene, being substituted for 3-(trifluoromethyl)benzoyl chloride, to prepare the title compound.

¹H NMR (400 MHz, d₆-DMSO) ppm 8.90 (s, 1H), 8.88 (dd, J=0.8, 2.5 Hz, 1H), 8.78 (s, 1H), 8.62 (dd, J=1.5, 4.8 Hz, 1H), 8.11 (ddd, J=0.8, 1.5, 7.8 Hz, 1H), 7.98 (s, 1H), 7.67 (t, J=1.8 Hz, 1H), 7.43-7.58 (m, 4H), 7.20 (d, J=2.2 Hz, 1H), 7.17 (t, J=8.1 Hz, 1H), 7.09 (dt, J=1.5, 7.6 Hz, 1H), 6.93 (ddd, J=0.8, 1.8, 8.1 Hz, 1H), 6.56 (ddd, J=0.8, 2.5, 8.1 Hz, 1H), 5.68 (s, 2H), 3.72 (s, 3H). MS (ESI) m/z=468 [M+H]+.

### Example 9

### N-[3-(4-Amino-7-pyridin-3-ylthieno[3,2-c]pyridin-3-yl)phenyl]-N′-phenylurea

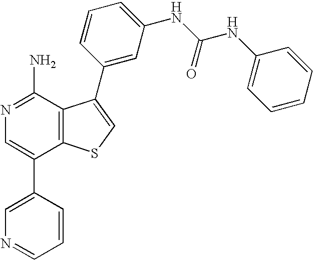

A procedure similar to Example 2 was used, with isocyanatobenzene being substituted for 3-(trifluoromethyl)benzoyl chloride, to prepare the title compound.

¹H NMR (400 MHz, d₆-DMSO) ppm 8.92 (s, 1H), 8.88 (dd, J=0.8, 2.3 Hz, 1H), 8.77 (s, 1H), 8.62 (dd, J=1.8, 4.8 Hz, 1H), 8.11 (ddd, J=0.8, 1.8, 7.8 Hz, 1H), 7.98 (s, 1H), 7.66 (t, J=1.8 Hz, 1H), 7.52-7.58 (m, 3H), 7.43-7.48 (m, 3H), 7.25-7.30 (m, 2H), 7.09 (dt, J=1.5, 7.6 Hz, 1H), 6.97 (tt, J=1.2, 7.6 Hz, 1H), 5.69 (s, 2H). MS (ESI) m/z=438 [M+H]+.

### Example 10

### N-[3-(4-Amino-7-pyridin-3-ylthieno[3,2-c]pyridin-3-yl)phenyl]-N′-[4-chloro-3-(trifluoromethyl)phenyl]urea

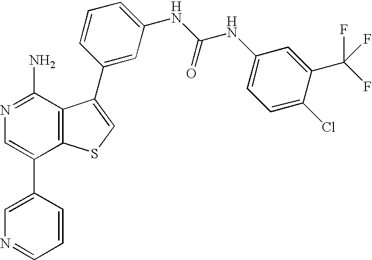

A procedure similar to Example 2 was used, with 1-chloro-4-isocyanato-2-(trifluoromethyl)benzene being substituted for 3-(trifluoromethyl)benzoyl chloride, to prepare the title compound.

¹H NMR (400 MHz, d₆-DMSO) ppm 9.28 (s, 1H), 9.11 (s, 1H), 8.88 (dd, J=0.8, 2.3 Hz, 1H), 8.62 (dd, J=1.5, 4.8 Hz, 1H), 8.09-8.12 (m, 2H), 7.98 (s, 1H), 7.54-7.68 (m, 6H), 7.47 (t, J=7.6 Hz, 1H), 7.13 (dt, J=1.5, 7.6 Hz, 1H), 5.68 (s, 2H). MS (ESI) m/z=540 [M+H]+.

### Example 11

### N-[3-(4-Amino-7-pyridin-3-ylthieno[3,2-c]pyridin-3-yl)phenyl]-N′-(2,6-difluorophenyl)urea

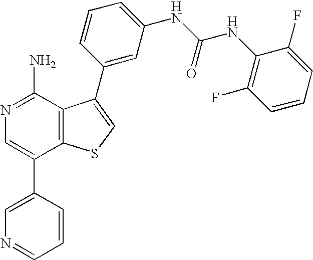

A procedure similar to Example 1 was used, with 1,3-difluoro-2-isocyanatobenzene being substituted for 3-(trifluoromethyl)benzoyl chloride, to prepare the title compound.

¹H NMR (400 MHz, d₆-DMSO) ppm 9.21 (s, 1H), 8.87 (dd, J=0.8, 2.3 Hz, 1H), 8.62 (dd, J=1.8, 4.8 Hz, 1H), 8.24 (s, 1H), 8.10 (ddd, J=1.8, 2.3, 7.8 Hz, 1H), 7.97 (s, 1H), 7.53-7.63 (m, 4H), 7.45 (t, J=7.8 Hz, 1H), 7.27-7.35 (m, 1H), 7.07-7.17 (m, 3H), 5.67 (s, 2H). MS (ESI) m/z=474 [M+H]+.

### Example 12

### N-[3-(4-Amino-7-pyridin-3-ylthieno[3,2-e]pyridin-3-yl)phenyl]-N′-benzylurea

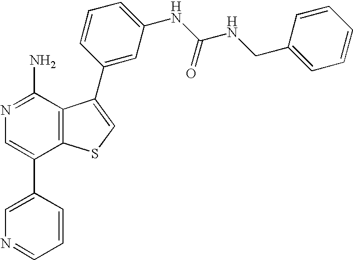

A procedure similar to Example 2 was used, with (isocyanatomethyl)benzene being substituted for 3-(trifluoromethyl)benzoyl chloride, to prepare the title compound.

¹H NMR (400 MHz, d₆-DMSO) ppm 8.87 (dd, J=0.8, 2.3 Hz, 1H), 8.82 (s, 1H), 8.62 (dd, J=1.5, 4.8 Hz, 1H), 8.10 (ddd, J=0.8, 2.5, 7.8 Hz, 1H), 7.96 (s, 1H), 7.61 (t, J=1.8 Hz, 1H), 7.47-7.57 (m, 3H), 7.40 (t, J=8.1 Hz, 1H), 7.22-7.38 (m, 5H), 7.01 (dt, J=1.5, 7.8 Hz, 1H), 6.74 (t, J=5.8 Hz, 1H), 5.67 (s, 2H), 4.30 (d, J=5.8 Hz, 2H). MS (ESI) m/z=452 [M+H]+.

### Example 13

### N-[3-(4-Amino-7-pyridin-3-ylthieno[3,2-c]pyridin-3-yl)phenyl]-IV-[4-(trifluoromethyl)phenyl]urea

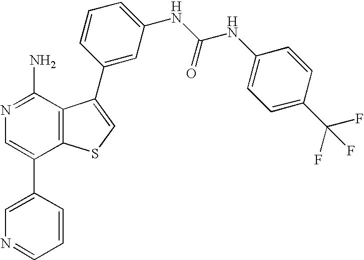

A procedure similar to Example 2 was used, with 1-isocyanato-4-(trifluoromethyl)benzene being substituted for 3-(trifluoromethyl)benzoyl chloride, to prepare the title compound.

¹H NMR (400 MHz, d₆-DMSO) ppm 9.22 (s, 1H), 9.06 (s, 1H), 8.88 (dd, J=0.8, 2.2 Hz, 1H), 8.62 (dd, J=1.8, 4.8 Hz, 1H), 8.11 (ddd, J=0.8, 2.5, 8.1 Hz, 1H), 7.98 (s, 1H), 7.54-7.69 (m, 8H), 7.48 (t, J=8.1 Hz, 1H), 7.13 (dt, J=1.5, 7.6 Hz, 1H), 5.69 (s, 2H). MS (ESI) m/z=506 [M+H]+.

### Example 14

### N-[3-(4-Amino-7-pyridin-3-ylthieno[3,2-c]pyridin-3-yl)phenyl]-N′-cyclohexylurea

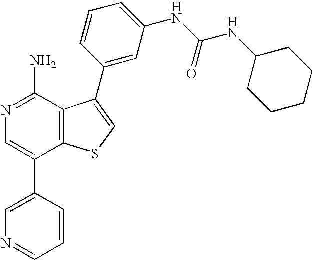

A procedure similar to Example 2 was used, with isocyanatocyclohexane, being substituted for 3-(trifluoromethyl)benzoyl chloride, to prepare the title compound.

¹H NMR (400 MHz, d₆-DMSO) ppm 8.87 (dd, J=0.8, 2.5 Hz, 1H), 8.62 (dd, J=1.5, 4.8 Hz, 1H), 8.53 (s, 1H), 8.10 (ddd, J=0.8, 2.5, 7.8 Hz, 1H), 7.97 (s, 1H), 7.53-7.57 (m, 3H), 7.36-7.46 (m, 2H), 6.99 (dt, J=1.5, 7.3 Hz, 1H), 6.16 (d, J=7.8 Hz, 1H), 5.67 (s, 2H), 3.42-3.48 (m, 1H), 1.12-1.81 (m, 10H). MS (ESI) m/z=444 [M+H]+.

### Example 15

### N-{3-[4-Amino-7-(3-pyridinyl)thieno[3,2-c]pyridin-3-yl]phenyl}-N′-(3,5-dimethyl-4-isoxazolyl)urea

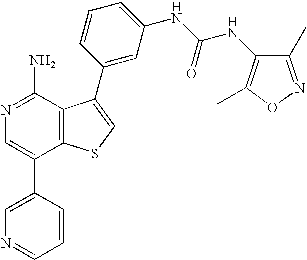

A procedure similar to Example 2 was used, with 4-isocyanato-3,5-dimethylisoxazole, being substituted for 3-(trifluoromethyl)benzoyl chloride, to prepare the title compound.

¹H NMR (400 MHz, d₆-DMSO) ppm 9.08 (s, 1H), 8.88 (dd, J=0.8, 2.2 Hz, 1H), 8.62 (dd, J=1.8, 4.8 Hz, 1H), 8.10 (ddd, J=0.8, 2.2, 7.8 Hz, 1H), 7.97 (s, 1H), 7.84 (s, 1H), 7.53-7.64 (m, 4H), 7.44 (t, J=8.1 Hz, 1H), 7.07 (dt, J=1.5, 7.8 Hz, 1H), 5.67 (s, 2H), 2.29 (s, 3H), 2.12 (s, 3H). MS (ESI) m/z=457 [M+H]+.

### Example 16

### N-{3-[4-Amino-7-(3-pyridinyl)thieno[3,2-c]pyridin-3-yl]phenyl}-N′-{4-[(phenylmethyl)oxy]phenyl}urea

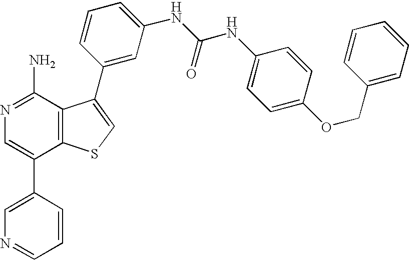

A procedure similar to Example 2 was used, with 1-isocyanato-4-[(phenylmethyl)oxy]benzene, being substituted for 3-(trifluoromethyl)benzoyl chloride, to prepare the title compound.

¹H NMR (400 MHz, d₆-DMSO) ppm 8.88 (dd, J=0.8, 2.5 Hz, 1H), 8.84 (s, 1H), 8.62 (dd, J=1.5, 4.8 Hz, 1H), 8.58 (s, 1H), 8.11 (ddd, J=0.8, 2.3, 7.8 Hz, 1H), 7.97 (s, 1H), 7.64 t, J=1.8 Hz, 1H), 7.30-7.58 (m, 11H), 7.07 (dt, J=1.5, 7.6 Hz, 1H), 6.93-6.96 (m, 2H), 5.69 (s, 2H), 5.06 (s, 2H). MS (EST) m/z=544 [M+H]+.

### Example 17

### 5-(4-Amino-7-pyridin-3-ylthieno[3,2-c]pyridin-3-yl)-N-[3-(trifluoromethyl)phenyl]indoline-1-carboxamide

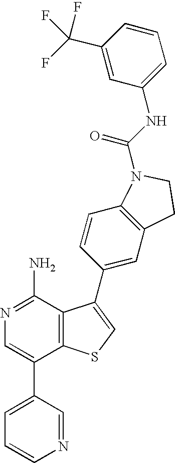

A procedure similar to Example 2 was used, with 1,1-dimethylethyl 5-(4,4,5,5-tetramethyl-1,3,2-dioxaborolan-2-yl)-2,3-dihydro-1H-indole-1-carboxylate being substituted for 1,1-dimethylethyl[3-(4,4,5,5-tetramethyl-1,3,2-dioxaborolan-2-yl)phenyl]carbamate and 1-isocyanato-3-(trifluoromethyl)benzene being substituted for 3-(trifluoromethyl)benzoyl chloride, to prepare the title compound.

¹H NMR (400 MHz, d₆-DMSO) ppm 8.95 (s, 1H), 8.88 (d, J=2.0 Hz, 1H), 8.62 (dd, J=1.5, 4.5 Hz, 1H), 7.90-8.17 (m, 5H), 7.25-7.57 (m, 6H), 5.68 (s, 2H), 4.25 (t, J=8.3 Hz, 2H), 3.29 (t, J=8.3 Hz, 2H). MS (ESI) m/z=532 [M+H]+.

### Biological Examples

A. Enzyme assays: Compounds of the present invention were tested against B-Raf in a fluorescence anisotropy binding assay. In general, the enzyme, fluorescent ligand, and test compound were allowed to come to equilibrium under conditions where there is a significant difference in the observed anisotropy, reflective of binding of the ligand to the enzyme, in the presence (>10×Kᵢ) or absence of test compound. The assay conditions were set so that the enzyme concentration is ≧1×K_{f} and the ligand concentration is less than the enzyme concentration.

Test compounds were serially diluted in DMSO and 0.1 μL was plated in low volume black 384-well plates. The assay was initiated by the addition of 10 L of an enzyme/ligand mix with a final assay composition of 50 mM HEPES (pH 7.3), 10 mM MgCl2, 1 mM CHAPS, 1 mM DTT, 1 nM fluorescent ligand, 2 nM competent B-Raf (competency determined as fraction of enzyme able to bind fluorescent ligand), and 0.169 nM-10 μM test compound. After incubation for two hours, the fluorescence anisotropy was read on a LJL Acquest with excitation at 485 nM and emission at 530 nM. Recombinant, His-tagged B-Raf (residues 462-770) that had been expressed in baculovirus was used for these experiments.

The data for dose responses were plotted as % Inhibition versus compound concentration following normalization using the formula 100*((U−C1)/(C₂−C₁)) where U is the unknown value, C1 is the average control value obtained for 1% DMSO, and C2 is the average control value for a known inhibitor. Curve fitting was performed with the equation y=A+((B−A)/(1+(10^{x}/10^{C})^{D})) where A is the y minimum, B is the y maximum, C is the log(XC₅₀), and D is the Hill slope. The results for each compound were recorded as pIC₅₀ values (—C in the above equation).

### Definitions:

Kᵢ=dissociation constant for inhibitor binding
K_{f}=dissociation constant for fluorescent ligand binding

The fluorescent ligand was the following compound:

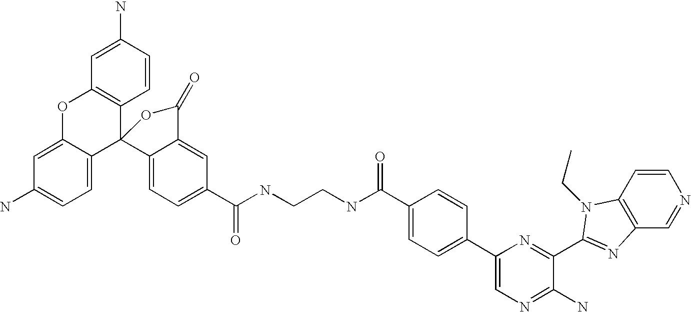

The exemplified Examples 1-17 were run in the recited cellular assay and each tested compound exhibited an pIC50 greater than 5.0.

B. Cellular assays: MEK1 phosphorylation by overexpressed B-Raf (V581E) Human B-Raf cDNA and human MEK1 cDNA were cloned by PCR, and inserted into expression vector pGene/V5-His (Invitrogen). Point mutation of each gene was induced by PCR mutagenesis. Both the substrate MEK1 (kinase inactive mutant, D208A) and B-Raf (V581E) were overexpressed by GeneSwitch system (Invitrogen) in GeneSwitch3T3 cells. GeneSwitch3T3 cells were cultured in low glucose DMEM (Sigma D6046) containing 10% fetal bovine serum (FBS), 100,000 units/Lpenicillin, 100,000 units/L streptomycin and 50 ug/ml hygromycine at 37° C. in a humidified 10% CO2, 90% air incubator. Cells were harvested using trypsin/EDTA, counted using a haemocytometer, and plated in a 96-well tissue culture plate (Becton Dickinson 354407) at 20,000 cells/well. After 6 hours, both B-Raf (V581E) and FLAG tagged MEK1 were transfected into cells by fugene 6 (Roche Diagnostics 1 814 443). Cells were incubated at 37° C., 10% CO₂ for 18-20 hours. The next day, cells were stimulated by mifepristone (Invitrogene), inducer of gene expression, for 4 hours at a final concentration of 10 nM. Compounds were diluted in DMEM at the final required concentration, from 10 mM stock solutions in DMSO. 100 uL/well of these dilutions were added to the each cell plates after removing medium. Medium containing 0.1% DMSO was added to control wells. After 2 hours, medium was removed by aspiration. Cells were lysed by ice cold lysis buffer (20 mM Tris-HCl pH8.0) containing 137 mM NaCl, 2 mM EDTA, 10% glycerol, 1% triton X-100, 1 mM NaF, 1 mM Na₃VO4 and protease inhibitors). Cell lysate was transferred to 96 well immunoassay black plate (Corning 3694), which were coated by 5 ug/ml of anti-FLAG antibody M2 (Sigma F3165) in PBS(−) and blocked by 5% BSA in PBST (PBS(−) containing 0.05% tween 20) for 2 hours. After overnight incubation, plates were washed 5 times with PBST and anti-phospho-MEK1/2 (Ser217/221) (Cell Signaling Technology) at 1:2000 diluted with 5% BSA in PBST was added. After incubation for 6 hours at room temperature, plates were washed 5 times with PBST, and HRP conjugated anti-rabbit IgG (Cell Signaling Technology) at 1:2000 diluted with 5% skim milk in PBST was added. After 1 hour incubation, plates were washed 5 times with PBST. For chemiluminescence detection, buffer was removed and BM chemiluminescence ELISA substrate (Roche Diagnostics 1 582 950) was added to wells. Following incubation for 3 min by mixing, chemiluminescence signal was measured using 1420 Multilabel Counter (Wallac).

The exemplified Examples 1-3, 5, and 8-14 were run in the recited cellular assay and each tested compound exhibited an IC₅₀ greater than 5.0.

1. A compound of formula (I): 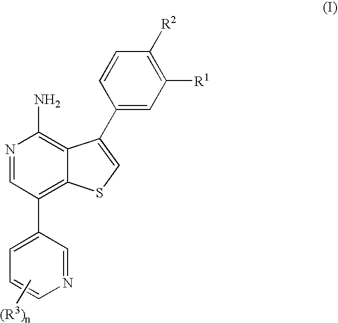
wherein:
R¹ is —N(H)—C(O)R⁴ or —N(H)—C(O)—N(H)R⁴ and R² is H; or
R¹ and R² together with the phenyl ring to which they are bonded form a moiety (i); 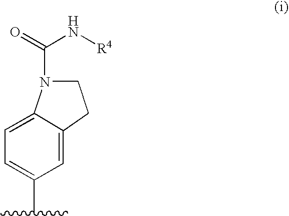
each R³ is independently selected from the group consisting of halo, alkyl, and haloalkyl;
R⁴ is selected from the group consisting of cyclohexyl; benzyl; unsubstituted, monosubstituted, or disubstituted phenyl wherein the substituent(s) is independently selected from the group consisting of halo, alkyl, haloalkyl, alkoxy, haloalkoxy, and benzyloxy; methylene-thienyl; and dimethyl-4-isoxazolyl; and
n is 0, 1, or 2;
or a pharmaceutically acceptable salt or solvate thereof.

2. The compound according to claim 1, wherein R¹ is —N(H)—C(O)R⁴.

3. The compound according to claim 1, wherein R¹ is —N(H)—C(O)—N(H)R⁴.

4. The compound according to claim 1, wherein R¹ and R² together with the carbon atoms to which they are bonded form a moiety (i).

5. The compound according to claim 1, wherein n is 0.

6. The compound according to claim 1, wherein R⁴ is unsubstituted phenyl.

7. The compound according to claim 1, wherein R⁴ is monosubstituted phenyl.

8. The compound according to claim 1, wherein R⁴ is monosubstituted phenyl wherein the substituent is selected from the group consisting of halo, haloalkyl, alkoxy, and benzyloxy.

9. The compound according to claim 1, wherein R⁴ is disubstituted phenyl wherein the substituents are halo and haloalkyl.

10. The compound according to claim 1, wherein R⁴ is disubstituted phenyl wherein the substituents are both halo.

11. The compound according to claim 1, wherein R⁴ is benzyl.

12. The compound according to claim 1, wherein R⁴ is cyclohexyl.

13. A compound selected from:
N-[3-(4-amino-7-pyridin-3-ylthieno[3,2-c]pyridin-3-yl)phenyl]-N′-[2-fluoro-5-(trifluoromethyl)phenyl]urea;
N-[3-(4-amino-7-pyridin-3-ylthieno[3,2-c]pyridin-3-yl)phenyl]-3-(trifluoromethyl)benzamide;
N-[3-(4-amino-7-pyridin-3-ylthieno[3,2-c]pyridin-3-yl)phenyl]-2-phenylacetamide;
N-[3-(4-amino-7-pyridin-3-ylthieno[3,2-c]pyridin-3-yl)phenyl]-2-(2-thienyl)acetamide;
N-[3-(4-amino-7-pyridin-3-ylthieno[3,2-c]pyridin-3-yl)phenyl]-2,5-difluorobenzamide;
N-[3-(4-amino-7-pyridin-3-ylthieno[3,2-c]pyridin-3-yl)phenyl]benzamide;
N-{3-[4-amino-7-(3-pyridinyl)thieno[3,2-c]pyridin-3-yl]phenyl}cyclohexanecarboxamide;
N-[3-(4-amino-7-pyridin-3-ylthieno[3,2-c]pyridin-3-yl)phenyl]-N′-(3-methoxyphenyl)urea;
N-[3-(4-amino-7-pyridin-3-ylthieno[3,2-c]pyridin-3-yl)phenyl]-N′-phenylurea;
N-[3-(4-amino-7-pyridin-3-ylthieno[3,2-c]pyridin-3-yl)phenyl]-N′-[4-chloro-3-(trifluoromethyl)phenyl]urea;
N-[3-(4-amino-7-pyridin-3-ylthieno[3,2-c]pyridin-3-yl)phenyl]-N′-(2,6-difluorophenyl)urea;
N-[3-(4-amino-7-pyridin-3-ylthieno[3,2-c]pyridin-3-yl)phenyl]-N′-benzylurea;
N-[3-(4-amino-7-pyridin-3-ylthieno[3,2-c]pyridin-3-yl)phenyl]-N-[4-(trifluoromethyl)phenyl]urea;
N-[3-(4-amino-7-pyridin-3-ylthieno[3,2-c]pyridin-3-yl)phenyl]-N′-cyclohexylurea;
N-{3-[4-amino-7-(3-pyridinyl)thieno[3,2-c]pyridin-3-yl]phenyl}-N-(3,5-dimethyl-4-isoxazolyl)urea;
N-{3-[4-amino-7-(3-pyridinyl)thieno[3,2-c]pyridin-3-yl]phenyl}-N-{4-[(phenylmethyl)oxy]phenyl}urea; and
5-(4-Amino-7-pyridin-3-ylthieno[3,2-c]pyridin-3-yl)-N-[3-(trifluoromethyl)phenyl]indoline-1-carboxamide;
and pharmaceutically acceptable salts and solvates thereof.

14. A pharmaceutical composition comprising a compound according to claim 1.

15. The pharmaceutical composition according to claim 14 further comprising a pharmaceutically acceptable carrier, diluent or excipient.

16. A method for treating a condition mediated by B-Raf kinase in a mammal in need thereof, said method comprising administering to the mammal a compound according to claim 1.

17. A method for treating a neurotraumatic condition in a mammal in need thereof, said method comprising administering to the mammal a compound according to claim 1.

18. A method for treating a susceptible neoplasm in a mammal in need thereof, said method comprising administering to the mammal a compound according to claim 1.

19. The method according to claim 18, wherein said susceptible neoplasm is selected from histiocytic lymphoma, melanoma, small cell lung cancer, non-small cell lung cancer, breast cancer, colon cancer, and pancreatic cancer.

20. A process for preparing a compound according to claim 1, said process comprising:
reacting a compound of formula (IV): 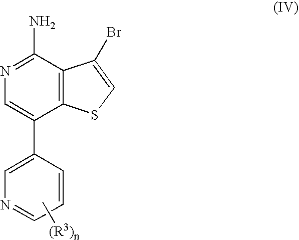
with a compound selected from formula (V), (VII) and (XII): 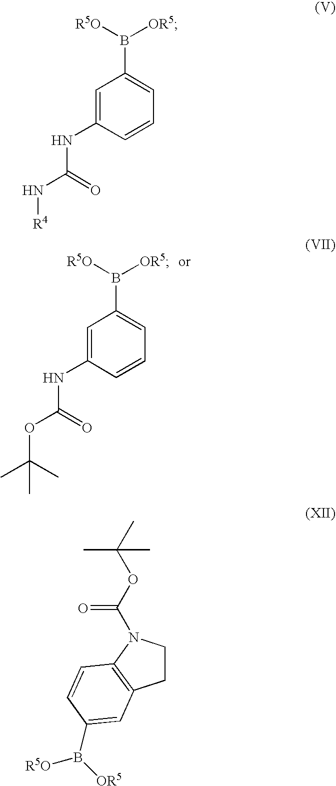
wherein R³, R⁴, and n are as defined in claim 1, and in each of compounds (V), (VII), and (XII), R⁵ is independently hydrogen or C₁ to C₆ alkyl, or the two R⁵ groups together with the boron and oxygen atoms to which they are bonded form a 5- or 6-membered ring, which is optionally substituted by 1 to 4 alkyl groups.

21. A compound according to claim 1 for use in therapy.

22. A compound according to claim 1 for use in the treatment of a condition mediated by B-Raf kinase in a mammal.

23. A compound according to claim 1 for use in the treatment of a neurotraumatic condition mediated by B-Raf kinase in a mammal.

24. A compound according to claim 1 for use in the treatment of a neoplasm susceptible to B-Raf kinase in a mammal.

25. The use of a compound according to claim 1 for the preparation of a medicament for the treatment of a condition mediated by B-Raf kinase in a mammal.

26. The use of a compound according to claim 1 for the preparation of a medicament for the treatment of a neurotraumatic condition in a mammal.

27. The use of a compound according to claim 1 for the preparation of a medicament for the treatment of a susceptible neoplasm in a mammal.

28. A pharmaceutical composition comprising a compound according to claim 1 for use in the treatment of a neurotraumatic condition in a mammal.

29. A pharmaceutical composition comprising a compound according to claim 1 for use in the treatment of a susceptible neoplasm in a mammal.
